(19) United States
(12) Patent Application Publication
(10) Pub. No.: US 2018/0148792 A1
(43) Pub. Date: May 31, 2018

(54) Method for Identification of a Deficient BRCA1 Function

(71) Applicants: T.S. SRIDHAR, Bangalore (IN); Nadathur Estates Pvt. Ltd., Bangalore (IN)

(72) Inventors: T.S. SRIDHAR, Bangalore (IN); Jyothi S. PRABHU, Bangalore (IN); Aruna KORLIMARLA, Bangalore (IN); José REMACLE, Jambes (BE)

(21) Appl. No.: 15/575,040

(22) PCT Filed: May 18, 2016
(86) PCT No.: PCT/IB2016/052911

§ 371 Date: Nov. 17, 2017

(30) Foreign Application Priority Data
May 19, 2015 (IN) 2515/CHE/2015

(51) Int. Cl. C12Q 1/6886; G01N 33/574

(52) U.S. Cl. CPC C12Q 1/6886; G01N 33/57415; G01N 2800/52; C12Q 2600/154; C12Q 2600/178; C12Q 2600/158

(57) ABSTRACT

The present invention is related to an in vitro method for identifying deficient BRCA1 function in a test biological sample obtained from a human individual, said method comprising the steps of:
Assaying the test sample to determine an expression profile of at least BRCA1 gene and ID4 gene; and
Establishing the ratio of BRCA1 to ID4 gene expression;
   wherein a BRCA1/ID4 ratio lower than a threshold value is used as positive criterion for identification of the deficient BRCA1 function in the tested sample.

### CROSS REFERENCE TO RELATED APPLICATIONS

This application is a filing under 35 U.S.C. 371 of International Application No. PCT/IB2016/052911 filed May 18, 2016, entitled “Method for Identification of a Deficient BRCA1 Function” which claims priority to Indian Patent Application No. 2515/CHE/2015 filed May 19, 2015, which applications are incorporated by reference herein in their entirety.

### FIELD OF THE INVENTION

The present invention is related to an in vitro method for predicting the presence of a deficient BRCA1 function in a biological sample obtained from a human patient.

### BACKGROUND

The linking of germ-line BRCA1/2 mutations to familial breast and ovarian cancer has been made more than 2 decades ago. Cancers with mutated BRCA1 and/or 2 BRCA1− are particularly sensitive to chemotherapy with Cisplatin or to irradiation. The reason is the formation of double stranded DNA breaks by these treatments which cannot be repaired appropriately in these cells since loss of BRCA 1 and BRCA2 impairs the Homologous-Recombination (HR) mediated Double-Strand Break (DSB) repair, and the cell undergo apoptosis.

It was found that treatment of BRCA1/2 mutated cells with PARP inhibitors (inhibition of the enzyme poly ADP ribose polymerase) resulted in chromosomal instability, cell cycle arrest and apoptosis (Farmer et al 2005, Targeting the DNA repair defect in BRCA mutant cells as a therapeutic strategy, nature, 434, p. 917-921). This therapeutic approach was called “synthetic lethality”. The combined treatment with PARP inhibitors and platinum salts has been proposed as a therapeutic strategy for BRCA mutated cancers (U.S. Pat. No. 8,143,241).

PARP inhibitors have been used in trials for patient with BRCA mutated. The US FDA approved in December 2014 the use of the PARP inhibitor Olaparib (Lynparza) along with the companion diagnostic “BRACAnalysis” for women with germ-line mutations in BRCA1/2 with advanced ovarian cancer.

PARP is involved in the repair of the single strand break (SSB). Inhibition of PARP activity leads to an accumulation of unrepaired single strand breaks that results in stalling and collapse of replication forks in replicative cells and, consequently, to DSB. This accumulation together with the lack of DSB repair in the presence of impaired BRCA1 and/or 2 leads to cell death. PARP inhibitors showed on the other side minimal toxic effect on cells having normal BRCA activity. Since in these patients, normal non mutant cells are heterozygous possessing one wild type allele, they are minimally affected by the presence of PARP inhibitors making this therapy rather safe. On the contrary, tumors predominantly show loss of heterozygosity explaining the selectivity of the PARP inhibitors on the BRCA1 and 2 deficient tumors.

The mechanism of PARP inhibitor-induced cytotoxicity could however be more complicated than simple synthetic lethality consequent to the disruption of two DNA repair pathways: Homologous Repair and Base excision Repair.

In 2004, Turner et al (Hallmarks of ‘BRCAness’ in sporadic cancers, Nat Rev Cancer, 4, p. 814-19) suggested that somatic disruption of the function of the same genetic pathways might be encountered in a sub-set of sporadic breast cancers, and they termed this phenotype, “BRCAness”. Collectively, the properties that define ‘BRCAness’ in sporadic cancers include a higher rate of proliferation, higher tumor grade, frequent mutational inactivation of TP53 and lack of expression of the luminal immunohistochemical markers (ER & PR) (Turner et al 2004). It was also noted that clinically Triple Negative Breast Cancers (TNBC), and the molecular class of Basal-like breast cancers (BLBC) have a significant phenotypic overlap with BRCA1-mutated tumors.

The excitement upon realizing the resemblence between familial BRCA1 mutated breast cancers and sporadic BLBC and TNBCs led to the hope that similar treatment might work in both sets of patients. The idea was that combined effect of DNA damage, tumor-specific DNA repair defects, and failure to stop or stall the cell cycle before the damaged DNA would be passed on to daughter cells leading to genome instability. This synthetic lethal therapeutic approach developed for germ line BRCA1 mutations was then tested on sporadic TNBC cancers supposedly having deficient BRCA1 (U.S. Pat. No. 8,143,241). However, the testing of this class of drugs in clinical trials for women with TNBCs gave disappointing results for multiple reasons. Right from the time of the coining of the TNBC category, it has been recognized that it had significant heterogeneity, but most trials did not include an appropriate molecular test to sub-stratify the patients. Even more embarrassingly the PARP inhibitor that was used in the largest unsuccessful phase III clinical trial of TNBCs.

Interestingly, a recent report by Hill et al 2014 (BRCA1 pathway function in basal-like breast cancer cells, Mol Cell Biol. 34, p. 3828-42) proposed that the non-compensated repair defect in sporadic BLBC cell-lines as opposed to BRCA1-mutated tumors, is the repair of stalled replication forks more so than HR mediated DSB repair. They provide proof that Cisplatin rather than PARP inhibitors are a better choice to treat sporadic BLBCs. In-vivo proof for this contention has been provided by the observations of Tassone et al 2009 (Loss of BRCA1 Function Increases the Antitumor Activity of Cisplatin against Human Breast Cancer Xenografts in Vivo, Cancer Biol Ther., 8, p. 648-53) who demonstrated the differential sensitivity of BRCA1-defective human breast cancer (HCC1937) xenografts in SCID mice to Cisplatin. However, the paucity of strong clinical proof, along with the challenge of significant toxicities (nephrotoxicity, myelosuppression and leukemogenesis) acted as a dampener for the wide-spread use of Cisplatin as a first line drug for the management of TNBCs. In addition, there is the concern of needless exposure, since most women with TNBCs are young and TNBCs are a heterogeneous group wherein only a fraction is thought to be deficient in BRCA1 function. The most encouraging study reported so far is the CALGB-40603 which reported a significant increase in the pathologic-complete response (pCR) rates to standard neoadjuvant Chemotherapy upon the addition of Carboplatin in women with stage II and III TNBC; 60% v 44%; P=0.001.

The presence or absence of BRCA1 protein in the cell nucleus is one of the methods used for the determination of BRCA1 deficiency. Such identification of BRCA1 protein is routinely performed by ImmunoHistoChemistry (IHC). It is one obvious method to estimate the level of BRCA1 in cells and in the nucleus in particular. However results are based on observation and are not always reliable (Pérez-vallés et al 2001, The usefulness of antibodies to the BRCA1 protein in detecting the mutated BRCA1 gene. An immunohistochemical study. J Clin Pathol., 54, p. 476-80). A detailed investigation of Milner et al 2013 (Validation of BRCA1 antibodiy MS110 and he utility of BRCA1 as a patient selection biomarker in immunohistochemical analysis of breast and ovarian tumors, Virchows Arch., 462, p. 269-79), concluded that BRCA1 expression levels in tissue samples is incompatible with the use of this protein as a statistically robust patient selection marker in immunohistochemistry. One of the limitations is that positive detection of the protein does not mean the BRCA1 is active. One of the explanations for these false positive results is the fact that antibody used for detection is directed against an epitope present in the N terminal of the protein. This portion of the protein is present in the non-full length splice variants of the proteins. So the positive signal can be due to the presence of inactive splice variants. So nuclear staining with the current BRCA1 antibodies, by itself cannot determine the BRCA1 adequacy.

BRCA1 expression has been proposed as a criterion for BRCAness. BRCA 1 expression compared to a reference has been proposed for selecting patients for neoadjuvant therapy (WO2009103784). BRCA1 expression was also proposed for determining the chemotherapeutic regimen for non-small-cell lung cancer (US20100009013). However, the BRCA1 expression value alone is not sufficient to account for BRCA1 deficiency since many ER+ breast cancers have low BRCA1 gene-expression but no evidence of increased genomic instability or poor clinical outcomes despite not being treated with chemotherapy. In the recent elegant analysis of all of the BLBCs in the The Cancer Genome Atlas (TCGA), Prat et al 2014 (Molecular features of the basal-like breast cancer subtype based on BRCA1 mutation status, Breast Cancer Res Treat., 147, p. 185-91.) demonstrated that even after excluding both germ-line and somatic BRCA1-mutated tumors and considering only wild-type BRCA1 containing BLBCs, they fall into two distinct classes based only on the basis of BRCA1 transcript levels.

BRCA1 enzymatic activity is difficult to assess in clinical specimens and presently there is no direct assay for its activity which can be used for routine analysis on clinical samples. Moreover, the clinical samples are usually fixed and embedded into formalin and all enzymatic activities are lost.

The expression of BRCA1 is subject to both transcriptional as well as epigenetic regulation. Its expression is on the average, two-fold lower in sporadic Triple Negative breast Cancers (TNBC) compared to ER-positive cancers. These regulatory factors were used as indications of BRCAness and for selection of patients for specific treatment based on the assumption of deficient DNA repair mechanism.

Promoter methylation was investigated as a possible mechanism of inhibition of the transcription and was found to be higher but only in a limited proportion of the TNBC cancers (around 15%). Recently Hsu et al 2013 (Methylation of BRCA1 Promoter Region Is Associated with Unfavorable Prognosis in Women with Early-Stage Breast Cancer. PLoS One, 8, p. 1-6) showed that BRCA1 promoter methylation detected by MSP (Methylation specific PCR), showed an unfavorable prognosis in women with early stage breast cancer. Turner et al 2007 (BRCA1 dysfunction in sporadic basal-like breast cancer, Oncogene, 26, p. 2126-32) also showed that the Basal like breast cancers had a higher percentage of BRCA1 promoter methylation. Lips et al 2011 (Indicators of homologous recombination deficiency in breast cancer and association with response to neoadjuvant chemotherapy. Ann Oncol., 22, p. 870-6) analyzed a series of 377 TNBCs from three different cohorts for BRCA1-like pattern by array Comparative Genomic Hybridization (aCGH), and BRCA1 promoter methylation. They found that 27% of TNBCs had promoter methylation at the BRCA1 locus.

Another alternate mechanism of BRCA1 gene down regulation was demonstrated by Turner et al (2007) by measuring transcript levels of ID4 and found ID4 as a negative regulator of the BRCA1. Its presence in higher levels was found in basal like breast cancer and resulted in repression of BRCA1 in those specimens. This was confirmed Wen et al 2012 (Id4 protein is highly expressed in triple-negative breast carcinomas: possible implications for BRCA1 downregulation, Breast Cancer Res Treat., 135, p. 93-102) who found a higher expression of ID4 in TNBC samples. ID4 is a member of the ID family of helix-loop-helix proteins, known to be key regulatory elements acting through negative regulation of gene transcription to block cell differentiation and they would rather stimulate the cell growth. The high expression of ID4 was proposed as a selection criterion for patient treatment in colorectal cancers. (US20130345144).

A microRNA, MIR182, is able to down regulate the expression of BRCA and to increase the sensitivity of cells to mutations. Treatment based on the increase level of microRNA is proposed as an alternative therapeutic (US20120059043).

Another proposed solution for the determination of BRCA1 deficiency was to use the gene signature of the tumors having BRCA1 or 2 mutations and then assesses the sporadic tumors in order to check for similar gene expression signatures (WO 2011153345, WO2011005384, WO2013124740, WO2012037378 and US2012/0225789 also proposed a list of marker genes for sub classification of cancers including the DNA repair deficient tumors. The limitation of the gene signature method is the large number of genes, (usually higher than 10 and even more than 30 and even more than 40 genes) needed to obtain a significant signature. A more specific signature limited to 10 genes has been proposed for identification of DNA repair deficient tumors (WO 2102/037378). The method shows significant variability in the level of expression due to the fact that some genes can vary according to many different factors. Also the method is based on the assumption of a linear relationship between the expression of these genes and the BRCA1 status either overexpressed or repressed in BRCA1 adequate samples or the opposite for the deficient BRCA1.

As noted by Turner and Ashworth 2011 (Biomarkers of PARP inhibitor sensitivity. Breast Cancer Res Treat., 127, p. 283-286) these studies are interesting proofs of principle, but validation of these signatures for use in the clinic is awaited. Another limitation of the gene signature was raised by Park 2010 (Clinical chemistry—The tragedy of the microarray anticommons, 56, p. 1682-85) who correctly stresses the fact that many genes have been individually protected making any new invention based on gene expression signature dependent on these previous patents so that the new invention would require many licenses before being commercially available.

Comparative genomic hybridization has also been proposed to identify change in loci number associated with the DNA repair deficiency or BRCA associated tumors (WO2011/048499). It was proposed that they represent a signature of BRCAness, and that such BRCA like profiles are a measure of BRCAness. Horlings et al 2010 (Integration of DNA copy number alterations and prognostic gene expression signatures in breast cancer patients. Clin Cancer Res, 16, p. 651-63) found that breast tumors from BRCA-mutation carriers show specific array Comparative Genomic Hybridization (aCGH) profiles which were also present in some sporadic breast cancer patients.

Patents applications WO 2014165785, WO2013096843 and WO20111160063 proposed an indirect method for the predicting patient response to damaging agents by the use of indicators of chromosome alterations comprising the loss of heterozygosity, the telomeric allelic imbalance and the large scale transition regions in chromosomes. The method is based on the assumption that these chromosomes alterations reflect the lack of repair of the cells which includes the deficiency of the BRCA1 enzyme. The method is indirect and requires quite a lot of DNA damages to be detected with confidence.

The loss of RAD1 was also proposed as an assay of defective HR mediated DSB repair. While the loss of RAD51 focus formation is a robust marker of HR deficiency in cell-lines, it has not been possible to perform this test reliably on clinical specimens in the routine lab setting.

All these studies are driven by the fact that there exists no direct assay of BRCA1 activity which could be used to estimate the BRCA1 efficiency in these tumors and to identify patients having the likelihood of BRCA1 deficiency. Even the use of the 5-IHC based “core-Basal” was not able to pick all tumors with BRCA1 deficiency.

So there is a need to provide a simple reliable assay to be used as companion-diagnostic for the selection of individuals having BRCA1 deficient cancer and to treat them taking profit of their limited capacity to repair DNA breaks.

### AIMS OF THE INVENTION

The invention aims to provide a method for the identification of deficient BRCA1 function in a test biological sample (tumor sample) obtained from a human individual which has deficient DNA repair and which does not present the drawbacks of the state of the art.

The invention also aims to use the method for selection of patients with deficient BRCA1 function and for the choice of adequate therapeutic agents related to the presence of deficient BRCA1.

### SUMMARY OF THE INVENTION

The present invention relates to the determination and the use of a single criterion and/or a score for identification of BRCA1 deficient sporadic cancers which have deficient DNA repair and the use of the method as companion diagnostic for selection of patients for treatment by DNA damaging agents including platinum-based chemotherapy alone or in combination with PARP inhibitors. There exists no simple test for the identification of sporadic BRCA1/2 deficient cancers.

The present invention relates to the development of quantitative selection criteria and a method to classify and identify BRCA1 deficient tumor and the use of such a classification to select patients who could benefit from therapies that rely on a lack or deficiency of HR DNA repair.

The present invention relates to an in vitro method for identifying deficient BRCA1 function in a test biological sample obtained from a human individual, said method comprising the steps of:
Assaying the test sample to determine an expression profile of at least BRCA1 gene and ID4 gene; and
Establishing the ratio of BRCA1 to ID4 gene expression;
   wherein a BRCA1/ID4 ratio lower than a threshold value is used as positive criterion for identification of the deficient BRCA1 function in the tested sample.

According to specific embodiments of the invention, the method comprises at least one or a suitable combination of the following features:
the threshold value for the BRCA1/ID4 ratio corresponds to the median value of a cohort of Triple Negative Breast Cancers (TNBC) samples;
the threshold value for the BRCA1/ID4 ratio is the value of said ratio separating a reference breast cancer population into a low and high ratio range wherein less than (about) 50% of the population is in the low ratio range, preferably less than (about) 32%, better less than (about) 16% and even better less than (about) 10%;
the BRCA1/ID4 ratio is lower than 1 and better lower than (about) 0.9 and even better lower than (about) 0.8, when the BRCA1 and ID4 gene expressions are calculated according to the same log scale;
the method further comprises the step of assaying the test sample to determine a percentage of cell nucleus positive for BRCA1 protein by immunohistochemistry, wherein a percentage lower than 20% and even lower than 10% and even lower than 5% is used as positive criterion for identification of a deficient BRCA1 function in the tested sample;
the method further comprises the step of assaying the test sample to determine the expression level of MIR182 microRNA, wherein an expression value higher than a threshold value is used as positive criterion for identification of a deficient BRCA1 function in the tested sample;
the criterion for identification of a deficient BRCA1 function further comprises: the expression value of MIR182 microRNA higher than a threshold value and the percentage of cell nucleus positive for BRCA1 protein, wherein the presence of at least two positive criteria among the said three criteria is used for identification of a deficient BRCA1 function in the tested sample;
the criterion for identification of a deficient BRCA1 function further comprises: the expression value of MIR182 microRNA higher than a threshold value, the percentage of cell nucleus positive for BRCA1 protein, the expression value of BRCA1 lower than a threshold value, the expression of ID4 higher than a threshold value and the methylation level of the BRCA1 promoter being higher than a threshold value, wherein the presence of at least two and better three and even better four positive criteria among the said criteria is used for identification of a deficient BRCA1 function in the tested sample;
the threshold value for MIR182 and/or ID4 expression is the value of said expression separating a reference breast cancer population into a low and high expression range wherein more than (about) 50% of the population is in the high expression range, preferably more than (about) 60%, better more than (about) 70% and even better (about) 84%;
a value is attributed to the criterion(a) used for identification of deficient BRCA1 function and the tumor is defined as deficient BRCA1 function if the score calculated on said value(s) by using a classifier algorithm reaches a threshold;
the in vitro biological test biological sample comprises cancer cells, preferably selected from the group consisting of breast cancer, ovarian cancer, early onset breast cancer, early onset ovarian cancer, sporadic breast cancer and sporadic ovarian cancer;
the method further comprises the step of the selection of patient(s) with deficient BRCA1 function and the step of determining therapeutic agent(s) comprising chemical or physical DNA damaging agent and/or PARP inhibitor, which would be suitable for treatment of said patient(s).
the threshold value for the positive criterion is determined to include more than (about) 30% and better more than (about) 50% and better more than (about) 75% and even better more than (about) 90% of patients selected according to the method of the invention who responded positively to chemical or physical therapeutic agent comprising DNA damaging agent and/or PARP inhibitor.

The present invention also relates to (the use of) the method of the invention (as companion diagnostic for) which further comprises the step for the selection of patient with deficient BRCA1 function and the step of determining chemical or physical therapeutic agent comprising DNA damaging agent and/or PARP inhibitor, which would be suitable (to be administrated) for treatment of these selected patients.

According to specific embodiments of the invention, (the use of) the method comprises at least one or a suitable combination of the following features:
the selected therapeutic agent comprises: platinum salt(s), alkylating agents, anthracyclines (such as doxorubicin) and others antitumoral compounds, such as bleomycin, cyclophosphamide, PARP inhibitor and irradiation agent derivatives thereof or a combination thereof;
the method further comprises the step of assaying the test sample to determine the presence of mutated P53 protein, wherein the presence of mutated P53 protein is considered as a positive criterion for treatment (with suitable active compounds, preferably the above identified compounds) of patients from which the test sample is obtained.

The invention results in a simple test that could be performed on Formalin-fixed, paraffin-embedded (FFPE) specimens in a clinical lab that is involved in routine molecular testing. The lab techniques are restricted to quantitative gene expression using TaqMan chemistry and in a particular embodiment to IHC.

### BRIEF DESCRIPTION OF THE DRAWINGS

FIG. 1 is a Venn diagram showing the distribution of tumor specimens among three criteria of deficient BRCA1 function in the 82 samples cohort when selected from the scoring based on these three criteria as described in example 3.

### DETAILED DESCRIPTION OF THE INVENTION

In the context of the present application and invention the following definitions apply.

“BRCA adequacy or BRCA functional” means a level of BRCA which is high enough in order to fulfill the process of DNA repair.

“deficient BRCA function or BRCA inadequacy or BRCAness or BRCA Non-Functional or BRCA dysfunction or BRCA inadequate” means a level of BRCA which is low for the enzyme to effectively repair damaged DNA.

BRCA1 is the main component of the DNA repair given his enzymatic activity for DSB DNA repair by homologous recombination (HR). BRCA deficiency (BRCAness) comprises BRCA1 deficiency and BRCA2 deficiency. BRCA2 is also involved into the DNA repair process. It regulates the RAD51 recombinase that mediates strand invasion and homology-directed repair.

Loss of BRCA1 function leads to a phenotype that reflects development from this cell of common origin down a basal-like pathway, as well as to phenotypes that are specific to BRCA1 tumors like being ER−. By contrast, BRCA2 tumors lack a clear pathological phenotype that is distinct from sporadic tumors, but show features of the underlying DNA-repair defect caused by loss of BRCA2 function. (Turner et al 2004).

The terms “ER+, PR+, HR+, HER2+, Triple negative Breast Cancer (TNBC) and Basal”, are commonly used in pathology to classified the breast cancers and are used in the patent as defined in Robbins and Cotran 2010 (Pathological Basis of disease, Elsevier, 8th Edition, p. 1066-1093). See also Perou 2011 (Molecular stratification of Triple-negative breast cancers, Oncologist, 16, 61-70) for the sub classification based on gene expression profiling.

The terms “ER−ve or ER+ve” means a tumor sample where the detection of estrogen receptor either as protein or as mRNA (ESRI) is null (or low) or positive (or high).

The terms “Triple Negative Breast Cancer or TNBC” refers to tumors being negative for the hormone receptors ER, PR and not having over-expression of HER2. Many of the TNBC are basal tumors and a sub-set of them are BRCA deficient.

The terms “Relative Normalization Unit or RNU” is a quantitative expression of the gene level in the samples based on the Ct determined by real time PCR and is calculated as proposed by Korlimarla et al 2014 (Separate quality-control measures are necessary for estimation of RNA and methylated DNA from formalin-fixed, paraffin-embedded specimens by quantitative PCR. J Mol Diagn., 16, p. 25.-260). It is comprised between (about) 0 and (about) 15 on a log 2 scale; the lower value is fixed at 1 and higher the RNU, higher the amount of gene expression.

The term “is used as positive criterion” means that the criterion is used alone or in combination with other criteria for the identification of deficient BRCA1 function in the tested sample. When the criterion is used alone, it means that if the criterion is fulfilled, the sample is considered as BRCA1 deficient. When the criterion is used with other criteria, it is one among multiple criteria and the sample is considered as BRCA1 deficient according to the mentioned conditions.

The term “the presence of 2, 3 or more positive criteria is used for identification of a deficient BRCA1 function” means that the when this condition is fulfilled, the sample is considered as BRCA1 deficient.

The term “reference breast cancer population” consist of all the breast cancers without exclusion. Its origin can be diverse like for example the TCGA data bank. It is preferably from a given country or given lab or given hospital where the test is performed.

The inventor found that it is possible to identify a tumor having BRCA deficiency based on one or very few parameters which can be obtained experimentally on a tissue sample even on a FFPE fixed tissue and quantified. The method is best adapted for identification of sporadic TNBC tumors with wild-type BRCA1 that have inadequate BRCA1 function and hence likely to respond to platinum therapy. The method is suited for classification of the tumors taking into account the bad or the good outcome of the patient treatment associated with the BRCA low function or dysfunction.

Preferably, the method of the invention is applied to an in vitro test biological sample obtained from a human individual, said sample comprising cancer cells selected from the group consisting of breast cancer, ovarian cancer, early onset breast cancer, early onset ovarian cancer, sporadic breast cancer and sporadic ovarian cancer.

Since BRCA1 expression alone is not sufficient to account for deficient BRCA1 function, other parameters need to be taken into consideration. Many of them have been proposed as presented in the background here above but they all have some limitations.

The inventors of the present invention found that the relative abundance of BRCA1 to the amount of ID4 transcripts in the same tumor expressed as a ratio of 2 log values is a far better discriminator of BRCA1 adequacy then any of the other parameters. This BRCA1/ID4 ratio has a very strong prognostic effect on the selection of samples to be classified as BRCA1 adequate or deficient.

In an embodiment, the BRCA1/ID4 ratio is calculated based on the transcript or mRNA abundance of the BRCA1 and ID4 genes.

In another embodiment, the ratio is calculated based on the amount and/or the concentration of the BRCA1 and ID4 proteins.

In a preferred embodiment, the biological sample is identified as deficient BRCA1 function when the threshold value for the BRCA1/ID4 ratio corresponds to the median value of a cohort of Triple Negative Breast Cancers (TNBC) samples. The median is the number separating the higher half of a data sample, here the ratio distribution in a TNBC cohort sample, from the lower half.

In a preferred embodiment, the biological sample is identified as deficient BRCA1 function when the threshold value for the BRCA1/ID4 ratio is the value of said ratio separating a reference breast cancer population into a low and high ratio range wherein less than (about) 50% of the population is in the low ratio range, preferably less than (about) 32%, better less than (about) 16% and even better less than (about) 10%. In other words for the 10%, the threshold is the value of the BRCA1/ID4 ratio separating 10% of a reference breast cancer population which has a low ratio from 90% which has a high ratio.

In a preferred embodiment, the BRCA1/ID4 ratio is lower than (about) 1 and better lower than (about) 0.9 and even better lower than (about) 0.8, when the BRCA1 and ID4 gene expressions are calculated according to the same log scale.

In another embodiment, the value for the BRCA1/ID4 expression ratio is comprised between about 0 and either about 40%, and better about 20% and even better about 16% and even better about 5% of the full scale of a reference breast cancer population.

In some embodiments, other criteria than the BRCA1/ID4 ratio have also to be fulfilled for the identification of deficient BRCA1 function.

In a preferred embodiment, the method for predicting the presence of a deficient BRCA1 function further comprises the step of assaying the test sample to determine a percentage of cell nucleus positive for BRCA1 protein by immunohistochemistry, wherein a percentage lower than about 20% and even lower than about 10% and even lower than about 5% is used as positive criterion for identification of a deficient BRCA1 function.

In another preferred embodiment, the method for predicting the presence of a deficient BRCA1 function further comprises the step of assaying the test sample to determine the expression level of MIR182 microRNA, wherein an expression value higher than a threshold value is used as positive criterion for identification of a deficient BRCA1 function. Preferably, the threshold value for MIR182 expression is the value of said expression separating a reference breast cancer population into a low and high expression range wherein at least (about) 50% of the population is in the high expression range, preferably at least (about) 60%, better at least (about) 70% and even better at least (about) 84%. In other word for at least (about) 84%, the threshold is the value of expression separating at least (about) 84% of a reference breast cancer population which has the high expression range from (about) 16% which has a low expression range. In another embodiment, the value of MIR182 expression is comprised between (about) 3%, better (about) 10%, even better (about) 40%, even better (about) 70% and the maximum (100% distribution) of the full scale expression MIR182 values of a reference population comprising of all intrinsic sub-types of breast tumors.

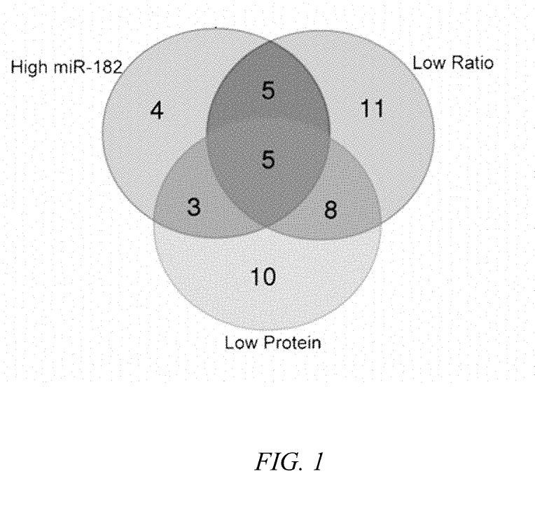

In a preferred embodiment, the sample is identified as BRCA1 deficient, if beside the BRCA1/ID4 ratio, other criteria are assayed comprising:
the expression value of MIR182 microRNA higher than a threshold value; and
the percentage of cell nucleus positive for BRCA1 protein;
   wherein the presence of at least two positive criteria among the 3 said criteria is used for identification of a deficient BRCA1 function in the tested sample. Preferably, the presence of all three positive criteria is used for (as) identification of a deficient BRCA1 function. Specific values for the selection based on 2 or 3 criteria in a tumor sample are given in FIG. 1 and example 3.

In another preferred embodiment, the sample is identified as BRCA1 deficient, if beside the BRCA1/ID4 ratio, other criteria are assayed comprising:
the expression value of MIR182 microRNA higher than a threshold value;
the percentage of cell nucleus positive for BRCA1 protein;
the expression value of BRCA1 lower than a threshold value;
the expression of ID4 higher than a threshold value; and
the methylation level of the BRCA1 promoter being higher than a threshold value;
   wherein the presence of at least two and better three and even better four positive criteria is used for (as) identification of a deficient BRCA1 function in a tested sample.

In a specific embodiment, the ID4 expression value higher than a threshold, is considered as positive criterion for identification of a deficient BRCA1 function. As an example, the threshold corresponds to the mean plus one standard deviation of the expression of ID4 in a reference cohort of samples.

In another specific embodiment, if the BRCA1 expression is lower than a threshold, it is considered as positive criterion for identification of a deficient BRCA1 function. As an example, the threshold corresponds to the mean less one standard deviation of the expression of BRCA1 in a reference cohort of samples.

In another specific embodiment, the tumor is identified as deficient BRCA1 function if both previous criteria (expression of ID4 and BRCA1) are also met together with the BRCA1/ID4 ratio. It is particularly true, if the threshold value for ID4 expression is the value of said expression separating a reference breast cancer population into a low and high expression range wherein at least (about) 50% of the population is in the high expression range, preferably at least (about) 60%, better at least (about) 70%, even better at least (about) 84% and even better at least (about) 90%.

The threshold value for the expression of BRCA1 is the value of said expression separating a reference breast cancer population into a low and high expression range wherein at least (about) 50% of the population is in the low expression range, preferably at least (about) 70%, better at least (about) 84% and even better at least (about) 90%.

In a particular embodiment, the value of ID4 expression is comprised between (about) 40%, better (about) 55%, even better (about) 70% and the maximum of the full scale expression ID4 values of a reference population of breast tumors when expressed on a log 2 scale.

In another embodiment, the value of ID4 expression is comprised between (about) 5%, better (about) 10%, even better (about) 40%, even better (about) 70% and the maximum of the full scale expression ID4 values of a reference population of breast tumors. The use of more stringent selection criteria will increase the specificity of the selection but will reduce the number of patients that will be eligible for treatment.

In a particular embodiment, the gene promoter preferably of BRCA1 and/or ESRI gene is significantly methylated. Methylation determination is preferably performed by Methyl light assay or by sequencing (Prabhu et al 2012, The epigenetic silencing of the estrogen receptor (ER) by hypermethylation of the ESRI promoter is seen predominantly in triple-negative breast cancers in Indian women. Tumour Biol., 33, p. 315-23). Methylation of promoter has an inhibitory effect on the transcription of the gene. In a reference set of breast tumors, 12% of the cancers were significantly methylated with the majority being Triple Negative tumors. Proportion of methylated TNBCs (18%) was more than two fold greater than that of HR positives samples (7%).

Preferably, beside de criterion of BRCA1/ID4 ratio, at least two and better three and even better four other criteria described here above are used to identify a deficient BRCA1 function.

In a preferred embodiment, the cancer sample is defined as BRCA1 deficient if at least two criteria are found positive among (1) BRCA1/ID4 ratio lower than a threshold value, (2) low or absence of protein in the cell nucleus and (3) MIR182 expression higher than a threshold value.

The invention also protects the identification of deficient BRCA1 function in tumor cells if it fulfils n−1 among the n criteria comprising the BRCA1/ID4 ratio lower than a threshold value and others criteria chosen among low or absence of protein in the cell nucleus, MIR182 expression higher than a than a threshold value, ID4 expression higher than a than a threshold value, BRCA1 expression lower than a threshold value, and high methylation of the BRCA1 promotor gene, with n being 3 or greater than 3.

Preferably, only four and better three and even better two criteria are used to identify deficient BRCA1 function the tested samples.

Preferably, the criterion used for defining the BRCA1 inadequacy of a tumor is exclusive from one another.

In particular, the criteria are applied in a sequential way and the already defined deficient samples are excluded from one criterion to the other.

In a preferred embodiment, a tumor is defined as deficient if a value is attributed to the criterion(s) used for identification of deficient BRCA1 function and the tumor is defined as deficient BRCA1 function if the score calculated on said value(s) by using an algorithm reaches a threshold.

Preferably, the algorithm is the sum of the different values or a mathematical equation. In a specific embodiment, each criterion is given a different weight in the calculation of the score or in the algorithm. Preferably, the BRCA1/ID4 ratio has the highest weight in the calculation of the score. Also the score calculation is a case by case non-linear weighting of the values.

In a specific embodiment, a value of zero or one is given for each parameter if they fulfil the condition for being associated respectively with BRCA1 deficiency of not and a score is calculated as the sum of the values. In still a preferred embodiment, three criteria are considered: the ratio BRCA1/ID4, the presence of BRCA1 protein in the nucleus and the level of MIR182 as proposed above and the value zero or one is given for each of them. The sample is considered as BRCA1 deficient if the score calculated as the sum of the values for the three criteria is one and even better if it is zero.

Preferably, the thresholds of selection for the different criteria mentioned above are the mean plus one standard deviation of said criteria in a reference breast cancer cohort.

In a particular embodiment, the gene expression value of the ID4 and MIR182 microRNA expression in a Log 2 scale is higher than a threshold being the mean value of their expression distribution in a reference breast cancer cohort. Even better the gene expression value of the ID4 expressed on a Log 2 scale is higher than the mean value plus 1 of the ID4 distribution of a reference breast cancer cohort.

In another embodiment, the gene expression value of the BRCA1 expressed in Log 2 scale is lower than a threshold being the mean value of the BRCA1 gene expression distribution of a reference breast cancer cohort.

In an embodiment, the tumor is defined as deficient BRCA1 function if the BRCA1/ID4 ratio expressed in Log 2 scale is lower than (about) 0.8 and better lower than (about) 0.6 and even better lower than (about) 0.5 as compared to the BRCA1/ID4 ratio distribution of a cohort of reference breast cancer population spanning between (about) 1 and (about) 15.

In a preferred embodiment, the method of the invention is used as companion diagnostic for selection of patient with deficient BRCA1 function and determining chemical or physical therapeutic agent comprising DNA damaging agent and/or PARP inhibitor, which would be suitable for treatment of said patients. Preferably, the therapeutic agent comprises: platinum salt, alkylating agents, doxorubicin, bleomycin, cyclophosphamide, PARP inhibitor and irradiation agent or a combination thereof.

In another preferred embodiment, the thresholds of the criteria are fixed to allow the selection of cancers being BRCA1 deficient and susceptible to respond to treatment by DNA damaging agents and/or PARP inhibitors.

Preferably, the threshold value for the positive criteria is determined to include at least 30% and better at least 50% and at least 75% and even better at least 90% of patients selected according to the method of the invention and who responded positively to the chemical or physical therapeutic agent comprising DNA damaging agent and/or PARP inhibitor.

Preferably, the threshold values for the criteria are fixed based on the outcome of patients treated with Platinum and/or PARP inhibitors. Also, the threshold values for the positive criteria are determined to include at least 50% and better at least 75% and even better at least 90% of the patients who do not relapse after at least (about) 3 years and better at least (about) 5 years after the treatment with Platinum and/or PARP inhibitors.

In a specific embodiment, the criteria of selection of deficient BRCA1 function and/or the thresholds are determined using a classifier algorithm. The classifier parameters are better determined by training set of tumor samples from patients who had a positive or negative outcome after treatment with Platinum and/or PARP inhibitors. Specifically, the classification is obtained by using a supervised method of classification, preferably a shrunken centroid like available in the PAM package in R software.

Another specific classifier is a centroid based method. Preferably the parameters of the classifier are optimized in order to obtain less than (about) 40% and better less than (about) 20% and even better less than (about) 10% misclassification in the training set.

More criteria are specifically added for the selection of deficient BRCA1 tumors. Such added criteria are chosen including, but not limited to, Ki67 and other genes associated with the cell division CENPF, CCNB1, ANLN, UBE2C, CCND1, BCL2 or other genes of interest comprising PgR, TFF1, GATA, GREB1, FOXA1, RAD51, AR or FANCA, BARD1, ATM.

Preferably, the determination assays related to the criteria of this invention are performed on Formalin-fixed, paraffin-embedded (FFPE) tissues. Fresh tissues are also part of the invention when available.

Preferably, the criteria are defined in comparison to a reference population of cancers comprising a random population of at least (about) 50 and better at least (about) 100 samples and determination of the population distribution and/or the scale between the lowest and the highest value for a given criterion. Preferably, the sample is a breast cancer or an epithelial ovary cancer (EOC).

In a preferred embodiment, the method further comprises the step of selecting patient(s) with deficient BRCA1 function and the step of determining therapeutic agent(s) comprising chemical or physical DNA damaging agent and/or PARP inhibitor, which would be suitable for treatment of said patient(s).

The therapeutic agent(s) are preferably selected from the group consisting of platinum salt(s), alkylating agents, antitumoral compounds especially anthracyclines, such as doxorubicin, bleomycin, cyclophosphamide, PARP inhibitor and irradiation agent or a combination thereof.

The present invention allows identification of deficient BRCA tumors potentially fitted for patient treatment with chemical and physical DNA-damaging agents including but not limited to chemicals reacting or forming adduct with DNA molecules, producing alkylation or intercalating into the DNA. Physical DNA damaging agents include irradiation with beta or gamma radiations. Irradiations induce multiple defects on DNA depending on type of radiation, the frequency and intensity of the radiations. This includes single strand or double strand breaks or interchain covalent links like thymine dimers, DNA deletion and oxidative base damages.

The DNA damaging drugs comprise: platinum salts as cis-platinum, oxaliplatin, carboplatin, to alkylating agents of the nitrogen mustards family as mechlorethamine, cyclophosphamide (Cytoxan), clhorambucil, ifosfamide or melphalan, of the nitrosoureas family including streptozocin, carmustine (BCNU) or iomustine, of alkyl sulfonates as busulfan, of the triazine family such as dacarbazine (DTIC) or temozolomide (Temodar) and of the ethylenimine family such as thiotepa or altretamine.

In still another embodiment, PARP inhibitors are selected efor treatment of the patient alone or preferably in association with the DNA damaging agents. Inhibitors comprise Olaparib (AZD2281), Rucaparib (AG-014699), Veliparib (ABT-888), niriparib (MK-4827) GPI-21016 (E7016) CEP 9722, MK-4827, BMN-673, BGB-290 and 3-aminobenzamid. Iniparib has been used but is now considered not to be acting as PARP inhibitor.

The treatment of the patient includes both the platinum based chemotherapy or irradiation and the PARP inhibitor as chemotherapeutic compound for treatment of the patient.

Other drugs acting as chemotherapeutic drugs are associated in particular treatments with the DNA damaging drugs and/or the PARP inhibitors and they comprise irinotecan hydrochloride, gemcitabine hydrochloride and Temozolomide. See for example Study of CEP-9722 in combination with Gemcitabine and Cisplatin used in clinical trial US-NLM Identifier NCT01345357. Other chemotherapeutic agents comprise the antimetabolites, antitumoral compounds, preferably anthracyclins (such as daunorubicin), topoisomerase inhibitors such as topotecan, mitotic inhibitors such as taxanes, and other targeted or hormone therapies.

Personalized medicine allows specific drugs to be provided for cancer treatments having specific features determined by a companion diagnostic. This is the case of the present invention.

Breast cancers having HER2+ receptor are one cancer type being treated by targeted therapies. Specific drugs acting on HER2+ tumors are available and have been shown to be effective in the treatment of the patient.

Patient with HER2+ cancer are usually primarily treated with chemotherapeutic drug like anthracyclin (such as doxorubicin) and with specific antiHER2 drugs (such as trastuzumab, pertuzumab, lapatinib, and ado-trastuzumab). It is also known that HER2+ tumors span all the spectra of BRCA1 expression and can be either HR+ve or HR−ve. Patients having HER2+ tumors are unlikely to benefit from Platinum salt or PARP inhibitor as proposed in this invention. In a specific embodiment, HER2+ tumors are not classified for BRCA1 inadequacy according to the present invention.

In an embodiment, the present invention is related to the selection of patients which would not benefit from target related specific treatments including but not limited to drug targeting ER or HER2 or FGFR receptor, PI3kinase, HER3, Tyrosine Kinase, mTOR, aromatase. In still another embodiment, the present invention is related to selection of patients with sporadic TNBC and who do not have mutation in BRCA1/2 genes.

In a preferred embodiment, the presence of mutated P53 protein is considered as a criterion or is required for the incorporation of the patient into the treatments related to deficient BRCA1 as proposed in this invention. The test sample is assayed to determine the presence of mutated P53 protein. Methods for determination of mutation are well known from the man of the art and comprising the sequencing, the PCR based and the array methods.

TNBC and basal breast cancers show a large proportion of samples where P53 protein is mutated. The loss of P53 results in weakening the control of the cell cycle and further accumulation of errors into the cell genome thus increasing the genome instability (Prat et al 2013, Genomic Analyses across Six Cancer Types Identify Basal-like Breast Cancer as a Unique Molecular Entity, Sci Rep., 18, p. 3544).

In another embodiment, the threshold value for the selection of deficient BRCA1 samples was fixed at 0.74 for the ratio BRCA1/ID4 gene expression, at 7.82 for ID4 and 8.32 for MIR182 when the gene expression values are expressed in term of RNU calculated as explained in Korlimarla et al (2014). The threshold value for the nucleus labelling by IHC was fixed at 10% of the cells.

Expressed in terms of proportion of samples, these thresholds correspond to 25% of the samples for the BRCA1/ID4 ratio, of 10% of the samples for ID4 and 16% of the samples for MIR182 when determined on a general reference cohort of 240 tumor samples obtained in 2 hospitals. Among the 240 tumors, 112 were HR+HER2−, 57 were HER2+, and 71 were TN. The threshold value for the nucleus corresponds to 32% of the samples determined on a cohort on 82 tumor samples from the previous cohort on which the assay for the nucleus was performed. This 82 cohort of samples was enriched in TN tumors compared to the original full cohort. Data on the samples are given in table 1.

Expressed in term of threshold on the scale of the expression value, the RNU threshold corresponds to 15% of the expression scale for the BRCA1/ID4 ratio, of 10% for ID4 and 5% for MIR182 when determined on a general reference cohort of 240 tumor samples obtained in 2 hospitals. The threshold value for the nucleus labelling by IHC corresponds to 10% of the labelling scale. The data are given in tables 2 and 3.

The present invention also covers (diagnostic) kits, means and reagents used in the present method of the invention, especially for the determination of the criteria needed for the identification of deficient BRCA1 function in breast or ovarian tumors.

### EXAMPLES

### Example 1. Human Breast Cancer Cohort and their Specifications

Surgically excised specimens of breast cancers were collected from two hospitals. An informed consent to use the material for research was obtained from all the patients and the study was approved by an ethics committee at each hospital. All samples were fixed in 10% neutral-buffered formalin at room temperature at the time of surgery and processed and stored as paraffin embedded blocks or FFPE samples. All samples were sectioned, stained with haematoxylin and eosin and only blocks with more than 50% cancer epithelial cells were used for molecular analysis. The specimens were collected over a four and a half year period between mid 2008 and early 2013.

The specimens used for this analysis (n=240) contained 57 HER2+(24%), 112 HR+HER2−ve (47%) and 71 TNBCs (30%). These 240 specimens are the ones that passed all QC criteria from a larger set of 340 specimens which in turn were collected from approximately 450 patients who comprise our complete cohort. The proportion of TNBCs in the samples used for this analysis is significantly higher (30 v 22) compared to the full cohort of 450 subjects. The characterization of the 183 samples (HR+HER2−ve and TNBCs) is presented in table 1.

### Immunohistochemistry

Immunohistochemistry (IHC) was done for BRCA1 according to standard procedures. Briefly, sections (5 t in thickness) were cut from FFPE blocks on poly L-lysine coated slides and subjected to deparaffinization in xylene and rehydrated in graded alcohol. After blocking endogenous peroxidase with a 3% hydrogen peroxide solution, antigen retrieval was done in 0.01M EDTA buffer at pH 8, in a microwave at 800 W for 2 min, 480 W for 7 min followed by 160 W for 11 min. Primary blocking was done with 3% bovine serum albumin (BSA, Sigma) for 30 min at room temperature. Primary antibody for BRCA1 (Clone MS110, Calbiochem Cat# OP92) at 1:100 dilution was applied for 1 hr at room temperature. Sections were further incubated with secondary antibody (DAKO REAL™ EnVision™) for 20 min as per the kit instructions, followed by development of the colour using DAB (DAKO REAL™ EnVision™) for 10 min. Sections were counterstained with hematoxylin and mounted after dehydration in graded alcohol and xylene. Appropriate positive and negative controls were run for each batch.

RNA and DNA Extraction and cDNA Conversion:

Methods to extract nucleic acids and conversion to cDNA were followed as outlined by Korlimarla et al (2014).

Gene Expression by qPCR and Normalization

The methods used for nucleic acid extraction, bisulphite conversion of the DNA, methylation assay, quantitative RT-PCR, selection of housekeeping genes (HKG) and quality control criteria for inclusion of samples in this analysis have been described (Prabhu et al 2012; Korlimarla et al 2014). All primers and probes for the mRNA assays were inventoried assays from Invitrogen.

The expression level of test genes was determined along with a panel of 3 reference genes (PUM1, RPL13A, ACTB). The reference genes normalize for any variations that may be introduced through variations in sample processing and handling methods which in turn lead to varied levels of RNA preservation in the FFPE blocks. Using 5 ng cDNA template per reaction real time PCR was done in duplicate using TaqMan qPCR chemistry on the Light Cycler 480 II (Roche Diagnostics). Total RNA Universal Human Reference RNA (Agilent, #740000) was also reverse transcribed and 1 ng of this template was run in the assay as a control. 5 ng cDNA from each sample is included in a total reaction volume of 10 μl. Pre-incubation and initial denaturation of the template cDNA was performed at 95° C. for 10 min, followed by amplification for 45 cycles at 95° C. for 15 sec and 60° C. for 1 min. All samples which had average Ct value of three housekeeping genes beyond 2 SDs above the mean were excluded from further analysis, as these specimens have very poorly preserved RNA.

Relative transcript abundance for the test gene were normalized to the mean Ct value of the three reference genes for each sample as ΔCt. The Relative Normalized Units (RNU) of expression of the test genes was calculated as 15−ΔCt. From each of the test genes in our study, we took the lowest value (x) of the 183 specimens, subtracted 1 from it and subtracted that value (x−1) from all the RNU values. This allowed us to obtain a series starting from 1 with no negative value for all transcript measurements. The results are expressed in RNU (Relative Normalized Units) on a scale of 1 to 15, with 1 being fixed as the lowest abundant gene. The dynamic range for transcript measurements was about 2¹² (4000 fold).

### MIR182 MicroRNA Assay

MicroRNA present in total RNA extracted as given above was converted to cDNA using Stem loop primers specific for the chosen miRNA according to published protocols, The TaqMan microRNA Reverse Transcription Kit (Applied Bio systems, #4366596) was used for cDNA conversion. Concentration of 50 ng/μl of total RNA was used for the conversion of microRNA to cDNA according to manufacturer's instructions using Verity 96 well thermal cycler (Applied Biosystems). Briefly, the reverse transcription reaction mixture was incubated at 16° C. for 30 minutes, 42° C. for 30 minutes, 85° C. for 5 minutes and finally held at 4° C.

TaqMan microRNA inventoried assays for qRT-PCR (Applied Biosystems, #4427975) were used for each of the test and control miRNA (RU48 and the test microRNA-MIR182). These assay kits comprise stem loop primers for cDNA conversion as well as TaqMan primer-probes for RT PCR analysis. 50 ng of total RNA was reverse transcribed for using control as well as test stem loop primers of a control microRNA, and then 2.5 ng of each microRNA-cDNA is included in a total PCR reaction mixture of 10 μl. Each microRNA analysis by qRT PCR was subjected to an enzyme activation step at 95° C. for 10 minutes, followed by 45 cycles of denaturation (95° C. for 15 seconds) and annealing (60° C. for 60 seconds). The assay IDs for each of the tested microRNAs was RNU48-001006 (control), hsa-miR-182-002334 (test).

### DNA Methylation Analysis

DNA was extracted from the interphase after RNA extraction from TRI Reagent according to the manufacturer's protocol. DNA was quantitated on a NanoDrop1000 spectrophotometer (Thermo Scientific). 500 ng of the DNA was bisulphite converted using the EZ DNA Methylation—Gold Kit (Zymo Research, Orange, Calif., USA, # D5005) according to the manufacturer's protocol.

QC measure to select for samples with adequate DNA after bisulfite modification were selected as outlined in previous publication. Korlimarla et al (2014). In brief, bisulphite converted DNA was amplified using Taqman primer probe specific for the ALU-C4 sequence as a control reaction for the amount of input DNA (Weisenberger et al 2005, Nucleic Acids Res., 33, p. 6823-36).

### Statistical Analysis

The normal distribution of continuous data was examined using Q-Q plots and histograms. Student t test was used to examine the relation between the gene markers tested when data was normally distributed. When not normally distributed Mann Whitney U test and Spearman's rank correlation were used. P values less than 0.05 was considered statistically significant. All analyses were performed using XLStat 2014 software.

TABLE I
Characterization of the breast cancer population of
183 samples of HR+ HER2− and TNBC.

| | HR+HER2−ve (%) | TNBC (%) |
|---|---|---|
| | n = 112 | n = 71 |
| IDC (%) | 93 | 93 |
| Median Age | 60 | 54 |
| Median tumor size (cm) | 3 | 3.5 |
| LN Positive | 73 (65) | 35 (49) |
| LN Negative | 33 (29) | 34 (48) |
| Grade I | 14 (12) | 3 (4) |
| Grade II | 53 (47) | 25 (35) |
| Grade III | 39 (36) | 39 (55) |
| Family history | | |
| <60 y age | 5 | 2 |

The determination of BRCA1 protein by IHC was performed on a subset of 82 samples of this cohort. It contained 55 Triple negative and 27 HR+HER2− tumors.

HER2+ cancers were not incorporated into the analysis. Basal cancers were identified based on the positive labeling of Cytokeratin 5/6 and/or EGFR by IHC.

### Example 2. Selection of Samples from a Cohort Based on the Use of One Criterion (BRCA1/ID4 Ratio)

82 samples for which the data on the presence of protein in the nucleus was available, were used in this analysis. The BRCA1 and ID4 expressions were obtained by the qPCR method of example 1. The 82 samples of the cohort were classified according to their BRCA1/ID4 ratio. They were considered as BRCA1 deficient if the BRCA1/ID4 ratio was lower than 0.74. 29 samples were found to meet this positive criterion and could be defined as BRCA1 deficient. All these 29 samples were TNBC and 16 were Basal by IHC. The threshold of the BRCA1/ID4 ratio was fixed as the median value of the ratio of the TNBC samples.

The selection of the samples based on a fixed value being a ratio of 2 log values represents a non-linear relationship between the two parameters (BRCA1 and ID4). First, the ratio was calculated on normalized values in which distributions of both gene expressions start at 1 and are on a log 2 scale. Sample defined as deficient BRCA1 function if the ratio is lower than 0.74, means that the relative level of ID4 is higher than the relative level of BRCA1. Moreover to be selected, the difference of Ct between the 2 values increases with the level of expression of BRCA1. For example, a sample with a low BRCA1 expression RNU of 3 will be selected if the RNU level of ID4 is 4 or higher. The minimal difference of RNU between the 2 samples is 1 RNU (log₂ scale) which means a difference of 2-fold on a linear scale. However In a specimen with high levels of BRCA1, for example a RNU of 9, the sample will be selected if the ID4 RNU is 12 or higher. In this case, the difference in value between the two transcripts is 3 (log 2 scale) which means a difference of 8-fold on a linear scale. This exemplifies the non-linear relationship in the selection of sample brought by the use of ratio: higher the expressed BRCA1, much higher must be the expression of ID4 for a sample to be selected as deficient.

### Example 3. Selection of Samples from a Cohort Based on the Use of Three Criteria (BRCA1/ID4 Ratio, MIR182 Expression, Cell Nucleus Positive for BRCA1 Protein) and a Scoring Calculation

In a preferred method for defining BRCA1 deficiency according to the present invention, the tumors were selected according to the three criteria determined according to the method presented in example 1 and shown as a Venn diagram in FIG. 1.

For MIR182 microRNA, the samples were considered as BRCA1 deficient if the expression of MIR182 was higher than a threshold value. The criterion of inclusion in the group of deficient BRCA1 function was to use the mean plus one standard deviation of a reference cohort of samples for the expression of MIR182 microRNA. We used a cohort of 183 samples to fix the threshold. According to this criterion, the samples with MIR182 expression RNU value higher than 8.32 were considered as deficient.

For the presence of protein in the nucleus, samples were considered as BRCA1 deficient if the percentage of cell nucleus positive for BRCA1 protein was lower than 10%.

The threshold for the BRCA1/ID4 ratio was the same as defined in example 2.

For each criterion, a value of 0 was given if it fulfils the BRCA1 deficient selection criterion and 1 if it did not fit with the criterion. A score was then calculated for each sample by addition of these 3 values. The score spans between 0 and 3. The score for samples having all the three criteria selected for deficient BRCA1 was 0 and the score for samples falling outside of all three criteria was 3. The samples having a score of 0 and 1 were selected and defined as BRCA1 deficient samples. 5 samples showed a score of 0 and 16 samples had a score of 1. A total of 21 samples were identified as BRCA1 deficient according to the three criteria and the scoring selection method used in this example. Out of these 21 samples, 20 were TNBCs and 1 was ER+ and 14 were of the IHC Basal subtype. The selection of samples with a score of 0 or 1 means that the selection of samples which comply with 2 or 3 of the parameters or in a general terms which comply with at least n−1 criteria, n being 3 in this case.

Selection based on the inclusion of the methylation of the BRCA1 promotor as a fourth criterion did not bring much new information. All the samples which were highly methylated were already selected by the three criteria described above.

### Example 4. Comparison of the Selection of Samples from a Cohort Based on the Use of One Criterion or with a Score Based on Three Criteria

A total of 21 samples were identified as BRCA1 deficient according to the three criteria and the defined score as proposed as proposed in example 3. Since 18 of the 21 samples defined as BRCA1 deficient selected by using the three criteria score were selected by a single criterion, the BRCA1/ID4 ratio, it appears that this criterion is a powerful indicator of the BRCA1 deficiency in a tumor.

The selection based on the two other criteria taken individually were less selective with 13 of the 17 samples selected for MIR182 alone were part of the 21 samples and 16 of the 26 samples selected for the nucleus labeling criteria alone were part of the 21 samples. Also, the selection comprised 3 and 2 ER+ samples respectively for the MIR182 and for the protein criterion, the other ones were TNBC. The relationship between the samples selected by the three criteria can be also visualized in a Venn diagram (FIG. 1). We can see that the inclusion of the MIR182 and BRCA1 protein nuclear presence criteria makes the selection more specific. 5 samples were common for the three criteria. Among the 21 selected based on three criteria, 18 were selected based on a low BRCA1/ID4 ratio, with 5 common only with MIR182 expression and 8 common only with low protein criterion; 3 were selected based on both MIR182 and low protein. These numbers clearly showed the major role played by the BRCA1/ID4 ratio in this selection.

Data on the number of samples selected according to individual criterion for the full breast cancer cohort of 240 samples or the 183 cohort (where the HER2+ have been removed) are presented in tables 2 and 3. The inclusion criteria for ID4 and BRCA1 expression was based on the determination of the mean plus one standard deviation of the gene expression in the 183 sample cohort. The range of the inclusion criteria values was also calculated on a linear scale and is presented in % in table 4.

TABLE 2
Number of samples being selected based on different
criteria in a full breast cancer cohort.

| Samples selected according to different | | |
|---|---|---|
| criteria for BRCA1 inadequacy | Number/total | % |
| BRCA1/ID4 ratio lower than 0.74 | 58/240 | 24.1 |
| ID4 gene expression higher than 7.82 | 27/240 | 11.2 |
| MIR182 gene expression higher than 8.32 | 31/240 | 12.9 |

The values are calculated based on a full cohort of 240 samples. Data are based on RNU values for the gene expression.

TABLE 3
Number of samples being selected based on different criteria
in a breast cancer cohort not including the HER2+ samples.

| Samples selected according to different | | |
|---|---|---|
| criteria for BRCA1 inadequacy | Number/total | % |
| BRCA1/ID4 ratio lower than 0.74 | 48/183 | 26.2 |
| ID4 gene expression higher than 7.82 | 26/183 | 14.2 |
| MIR182 gene expression higher than 8.32 | 26/183 | 14.2 |
| Nucleus labeling lower than 10% for BRCA1 | 26/82 | 31.7 |
| by IHC | | |

The values are calculated based on a cohort of 183 samples or of 82 samples for which BRCA1 protein nucleus detection by IHC labeling was available. The data are based on RNU values for the gene expression.

TABLE 4
Range of inclusion criteria values calculated on a liner scale

| | Scale limits | Scale range |
|---|---|---|
| Criteria of selection | (linear) | selection in % |
| BRCA1/ID4 ratio lower than 0.74 | 0-2.53 | 0-29 |
| Nucleus labeling lower than 10% for | 0-100 | 0-10 |
| BRCA1 by IHC | | |
| ID4 gene expression higher than 2^{3.9} | 0-1314 | 6.7-100 |
| from the maximum value | | |
| MIR182 gene expression higher than | 0-2732 | 4.4-100 |
| 2^{4.5} from the maximum value | | |

The values are calculated using a selection threshold data based on RNU values for the gene expression on a 183 cohort of cancers being HR+ and TNBC (not including the HER2+) and the % of labeled nuclei as explained in example 2 and 3.

### Example 5 Biological and Clinical Data of the Selected Samples

The BRCA1 dysregulation was described as strongly related to the degree of basal phenotype Turner et al, (2004, 2007). This observation was known for the cancers of germline BRCA1 mutations and was extended for the sporadic breast cancers.

The expressions of 3 genes (FABP7, KRT6B, DSC) known to be associated with the basal like cancers were tested on the samples being either defined as deficient or adequate. TNBC alone were selected in order to allow comparison of the data. The results are presented in table 5. They are expressed as the RNU of the qPCR assays as defined previously for the other genes. The results showed that the 3 genes were higher expressed in the average in the deficient samples compared to the adequate in concordance with the higher selection of basal like cancers in the deficient group.

BRCAness is also associated with a high number of Triple Negative (ER−, PR− and HER2−) cancers. Of the 21 selected samples for BRCA1 inadequacy based on 3 parameters with a scoring selection, 20 were TNBC with one being ER+. Also very informative, all the 29 samples selected on the criterion of low BRCA1/ID4 ratio alone were TNBC with no ER+. This high number of TNBC in the selection is in accordance with the original definition of BRCAness by Turner et al (2004).

TABLE 5
Mean values of the expression of 3 genes known to be associated
with basal cancers in the deficient and adequate TNBC samples.

| | FABP7 | KRT6B | DSC |
|---|---|---|---|
| BRCA1 deficient | 11.6 | 10.6 | 13.8 |
| BRCA1 adequate | 11.0 | 9.9 | 11.9 |

The gene expressions (RNU) were determined on samples defined as deficient or adequate from a cohort of 55 TNBC based on the use of the three criteria score as defined in example 3.

### Example 6 Definition and Selection of BRCA1 Adequate Samples

The definition of BRCA1 adequate was based on the use of two criteria: BRCA1 gene expression >9 or BRCA1/ID4 ratio >1. The 82 samples of the cohort were classified according to these two criteria. The results were the following: 18 samples were positive and selected for the BRCA1 gene expression criterion and 39 were positive for the BRCA1/ID4 ratio. In total, 39 were selected as adequate.

The full cohort of the 82 samples was then divided into adequate, deficient BRCA1 and undefined samples. When taking the BRCA1/ID4 ratio as criterion for deficient BRCA1, the respective numbers of samples falling into these 3 categories were respectively 29, 39 and 14. So globally 68 of the 82 samples were classified, the other 14 samples were considered as undetermined for BRCA1 deficiency.

When the three criteria and the scoring were used for the identification of the deficient BRCA1 function, the number of samples falling into these 3 categories were respectively 21, 39 and 22. According to these classification criteria, globally 60 of the 82 samples were classified, the other 22 samples were considered as undetermined for BRCA1 deficiency.

1. An in vitro method for identifying deficient BRCA1 function in a test biological sample obtained from a human individual, said method comprising the steps of:
Assaying the test sample to determine an expression profile of at least BRCA1 gene and ID4 gene; and
Establishing the ratio of BRCA1 to ID4 gene expression;
wherein a BRCA1/ID4 ratio lower than a threshold value is used as positive criterion for identification of the deficient BRCA1 function in the tested sample; and
wherein the BRCA1/ID4 ratio is lower than 1 when the BRCA1 and ID4 gene expressions are calculated according to the same log scale.

2-3. (canceled)

4. The method of claim 1, wherein the BRCA1/ID4 ratio is lower than 0.9, when the BRCA1 and ID4 gene expressions are calculated according to the same log scale.

5. The method of claim 1, which further comprises a step of assaying the test sample to determine a percentage of cell nucleus positive for BRCA1 protein by immunohistochemistry, wherein a percentage lower than 20% is used as a further positive criterion for identification of a deficient BRCA1 function.

6. The method of claim 1, which further comprises a step of assaying the test sample to determine the expression level of MIR182 microRNA, wherein an expression value higher than a threshold value is used as a further positive criterion for identification of a deficient BRCA1 function in the tested sample.

7. The method according to claim 1, wherein the criterion for identification of a deficient BRCA1 function further comprises:
a step of assaying the test sample to determine an expression level of MIR182 microRNA, wherein the expression value of the MIR182 microRNA is higher than the threshold value and is used as positive criterion for identification of a deficient BRCA1 function in the tested samples; and
a step of assaying the test sample to determine a percentage of cell nucleus positive for BRCA1 protein by immunohistochemistry, wherein a percentage lower than 20% is used as positive criterion for identification of a deficient BRCA1 function; and
wherein the presence of at least two positive criteria among the said three criteria is used for identification of a deficient BRCA1 function in the tested sample.

8. The method according to claim 1, wherein the criterion for identification of a deficient BRCA1 function further comprises:
a step of assaying the test sample to determine an expression level of MIR182 microRNA, wherein the expression value of the MIR182 microRNA is higher than the threshold value and is used as positive criterion for identification of a deficient BRCA1 function in the tested sample;
a step of assaying the test sample to determine a percentage of cell nucleus positive for BRCA1 protein by immunohistochemistry, wherein a percentage lower than 20% is used as positive criterion for identification of a deficient BRCA1 function;
the expression value of BRCA1 lower than a threshold value;
the expression of ID4 higher than a threshold value; and
the methylation level of the BRCA1 promoter being higher than a threshold value; and
wherein the presence of at least two positive criteria among the said criteria is used for identification of a deficient BRCA1 function in the tested sample.

9. The method according to claim 8, wherein the threshold value for MIR182 and/or ID4 expression is the value of said expression separating a reference breast cancer population into a low and high expression range wherein more than 50% of the population is in the high expression range.

10. The method according to claim 1, wherein a value is attributed to the criterion(a) used for identification of deficient BRCA1 function and the tumor is defined as deficient BRCA1 function if the score calculated on said value(s) by using a classifier algorithm reaches a threshold.

11. The method of claim 1, wherein the in vitro test biological sample comprises cancer cells selected from the group consisting of breast cancer, ovarian cancer, early onset breast cancer, early onset ovarian cancer, sporadic breast cancer and sporadic ovarian cancer.

12. The method according to claim 1, which further comprises the step of selecting patient(s) with deficient BRCA1 function and the step of determining therapeutic agent(s) comprising chemical or physical DNA damaging agent and/or PARP inhibitor, which would be suitable for treatment of said patient(s).

13. The method according to claim 12, wherein the therapeutic agent(s) are selected from the group consisting of platinum salt(s), alkylating agents, antitumoral compounds.

14. The method according to claim 12, which further comprises the step of assaying the test sample to determine the presence of mutated P53 protein, wherein the presence of mutated P53 protein is considered as a positive criterion for treatment of patients from which the test sample is obtained.

15. The method of claim 1, wherein the threshold value for the positive criterion is determined to include more than 30% of patients who responded positively to chemical or physical therapeutic agent comprising DNA damaging agent and/or PARP inhibitor.

16. The method according to claim 13, wherein the therapeutic agent comprises anthracyclines.

17. The method according to claim 13, wherein the therapeutic agent(s) are selected from the group consisting of doxorubicin, bleomycin, cyclophosphamide, PARP inhibitor and irradiation agent or a combination thereof.
